(19) United States
(12) Patent Application Publication
(10) Pub. No.: US 2024/0358023 A1
(43) Pub. Date: Oct. 31, 2024

(54) Agricultural Composition and Method of Making the Same

(71) Applicant: Active Micro Technologies, LLC, Lincolnton, NC (US)

(72) Inventors: Durant Scholz, Lincolnton, NC (US); Tiffany Russell, Bessemer City, NC (US); Erica Batounis, Lincolnton, NC (US); Maureen Drumwright, Charlotte, NC (US)

(73) Assignee: Active Micro Technologies, LLC, Lincolnton, NC (US)

(21) Appl. No.: 18/644,744

(22) Filed: Apr. 24, 2024

Related U.S. Application Data
(60) Provisional application No. 63/462,371, filed on Apr. 27, 2023.

(51) Int. Cl. A01N 63/20; A01N 63/50; A01N 65/44; A01P 21/00; C12P 7/6436; C12P 21/00

(52) U.S. Cl. CPC A01N 63/20; A01N 63/50; A01N 65/44; A01P 21/00; C12P 7/6436; C12P 21/00

(57) ABSTRACT

A method for producing a biostimulant by fermenting bacteria from the Lactobacillaceae family in a growth media containing Spartina alterniflora and extracting active compounds, and an agricultural composition comprising the biostimulant produced thereof.

### CROSS-REFERENCE TO RELATED APPLICATION

The present application claims priority from U.S. Provisional Application No. 63/462,371 filed on Apr. 27, 2023, the disclosure of which is incorporated by reference herein.

### TECHNICAL FIELD

The present disclosure relates to an agricultural composition and method of making the same, and more specifically, a plant biostimulant to promote healthy plant growth and improve agricultural efficiency. The plant biostimulant includes a fermentation product derived from the fermentation of a gram-positive bacteria from the family of Lactobacillaceae.

### BACKGROUND OF THE DISCLOSURE

Agricultural livelihoods contribute to food security, economic stability, and global sustenance. As the backbone of trade, agriculture affects international and domestic interactions from corporation levels to local farms. As sustainability concerns rise, alternative methods of plant cultivation become essential in agriculture such as the use of biostimulants that offer faster harvest cycles, minimize water consumption, and maximize control over growing conditions. Biostimulants are one of the latest technological advancements in modern agriculture. They are beneficial substances that improve the plant's nutrient conditions for a better plant size and crop yield. There is not yet an agreed upon definition in the industry, but the 2018 Farm Bill defines biostimulant as any “substance or microorganism that, when applied to seeds, plants, or the rhizosphere, stimulates natural processes to enhance or benefit nutrient uptake, nutrient efficiency, tolerance to abiotic stress, or crop quality and yield.” Biostimulants act on different mechanisms than fertilizers, and are not crop protectants because they improve plant vigor without affecting pathogens. The last few decades have seen a substantial increase in the research and use of bio-stimulants. They improve the plant's ability to withstand numerous stresses, such as high salt concentrations in the soil, drought, flooding, and nutrient based plant disorders.

The plant microbiome plays a crucial role in plant growth, development, and stress resilience. The natural microbiome surrounding a plant is capable of facilitating plant growth by improving biomass, supporting nutrient uptake, and improving soil nutrient conditions for better crop yield. Plants actively recruit microorganisms from surrounding microbial reservoirs, including microorganisms from the soil and phyllosphere bacteria used by above-ground plants. The greatest concentration of microorganisms is typically found in the plant rhizosphere as a direct response to the plant root commonly expelling carbon that is fixed through photosynthesis. Plants colonized by the plant growth promoting bacteria have a greater plant fitness and are capable of decreasing the deleterious effects of phytopathogens on plants.

Crop production has been facing many challenges in recent years such as climate change, drought, demographic development, and the increasing demand for sustainable crop production. Due to these challenges, the creation on synthetic microbiomes have become an area of interest because of the importance the plant microbiome plays in plant growth and crop efficiency. There have been recent studies to create biostimulants and synthetic organisms that enhance the beneficial microbiome located in and around the plant and soil. However, these biostimulants and synthetic organisms are not able to compete with the natural residential microflora that currently exists in the field. Furthermore, the synthetic organisms created are not multifunctional and are not able to perform all of the characteristics that natural microorganisms are capable of, such as improving plant tolerance to environmental stress and improving plant nutrition. Therefore, there is a need in the art for the creation of a multifunctional plant biostimulant capable of improving soil nutrient conditions for better plant mass, enhancing the beneficial microbes in the plant microbiome, and improving abiotic stress tolerance.

Beneficial bacteria encompass a major category of plant biostimulants. The most prominent group of this category are plant growth-promoting rhizobacteria that colonize plant rhizospheres. They are capable of improving plant growth, improving mineral uptake, controlling pathogens, and increasing resistance to abiotic stress. It is known that beneficial bacteria from the Lactobacillaceae family are also capable of functioning as a plant biostimulant; however, when members of the Lactobacillaceae family are used as a biostimulant, the compositions must consist of intact live organisms as discussed in WO2020191508A1. Lysed ferments created from the Lactobacillaceae family may also be used as plant biostimulants; however, the fermentation mixture must consist of both a yeast and bacteria as discussed in U.S. Pat. No. 9,131,700B2 or a second fermentation method must be applied as discussed in CA3011849A1. Therefore, there is a need in the art for improved methods of creating plant biostimulants and biostimulants made from the same.

### BRIEF SUMMARY OF THE INVENTION

In one aspect, the present discloses relates to a method of producing a plant biostimulant, which may comprise adding at least one stalk, stem, or seed of Spartina alterniflora to a growth media to form a fermentation medium, adding at least one cell of Lactobacillaceae to the fermentation medium to form a mixture for fermentation, fermenting the Lactobacillaceae in the mixture for fermentation to produce a fermentation product; and applying a filtration method to the fermentation product to extract a plant biostimulant from the fermentation product.

The plant biostimulant may comprise lipopeptides having 2-7 amino acids optionally coupled with an alkyl group having a C1-C18 chain.

The growth media may comprise at least one selected from peptone, dextrose, ammonium citrate, sodium acetate, magnesium sulfate, manganese sulfate, or potassium phosphate.

The fermentation step may be performed at a temperature in a range of about 15° C. to about 25° C.

The fermentation step may be performed for a time period of 12 hours to about 18 hours.

The fermentation step may be performed at a pH range of about 4.0 to 6.7.

The Spartina alterniflora may be included in an amount of about 1 wt % to about 20 wt %, based on a total amount of the fermentation medium.

The Lactobacillaceae is present in an amount of about 0.5 wt % to about 1 wt %, based on a total amount of the mixture for fermentation.

The Lactobacillaceae is present in a concentration of about 10⁶ CFU/ml to about 10⁸ CFU/ml in the mixture for fermentation.

In one aspect, the present disclosure relates to a method of improving nutrient uptake in a plant to stimulate increase in plant mass, which may comprise applying a plant biostimulant to a plant, wherein the plant biostimulant may comprise an extract obtained from a fermentation product of a gram-positive bacteria from the family of Lactobacillaceae and a growth media comprising Spartina alterniflora.

The plant biostimulant may be applied by spraying the plant above the root with the plant biostimulant, applying the plant biostimulant to the roots of the plant, applying the plant biostimulant to a soil the plant is disposed in, applying the plant biostimulant to a medium the plant is disposed in, applying the plant biostimulant to a seed prior to growing the plant, or applying the plant biostimulant to leaves of the plant.

In one aspect, the present disclosure relates to a plant biostimulant, which may comprise an extract obtained from a fermentation product of a gram-positive bacteria from the family of Lactobacillaceae and a growth media comprising Spartina alterniflora.

In one aspect, the present disclosure relates to an agricultural composition comprising the plant biostimulant.

The agricultural composition may further comprise water, where the plant biostimulant may have a concentration of about 0.1 wt % to about 10 wt % in the agricultural composition.

In one aspect, the present disclosure relates to a method of improving nutrient uptake in a plant to stimulate increase in plant mass, which may comprise applying the agricultural composition to a plant.

### BRIEF DESCRIPTION OF DRAWINGS

The present disclosure may be best understood by reference to the following description taken in conjunction with the accompanying drawings. The present disclosure should not be construed as being limited to the drawings.

FIG. 1 depicts a system used to grow the plants of the Example, the Comparative Example, and the Control.

FIG. 2 depicts the results of tomato biomass in the Experimental Example for each of the Example, the Comparative Example, and the Control.

FIG. 3 depicts root size after daily treatment of the agricultural composition of the Example.

FIG. 4 depicts root size after bi-weekly treatments of the agricultural composition of the Example.

FIG. 5 depicts root size after weekly treatment of the agricultural composition of the Example.

FIG. 6 depicts the root size of the Control.

### DETAILED DESCRIPTION OF THE INVENTION

The present disclosure provides a novel method for creating a biostimulant using bacteria from the Lactobacillaceae family by fermenting the bacteria in a growth media that comprises Spartina alterniflora and extracting active compounds to create a more effective biostimulant.

Spartina alterniflora, commonly known as smooth cordgrass, is a perennial grass found in intertidal estuaries and salt marshes. Spartina alterniflora spreads by vegetative propagation using underground rhizomes, as well as through seeds. It is an effective soil stabilizer and uses its root-rhizome system to reduce soil erosion. Spartina alterniflora is extremely tolerable to abiotic stresses such as high salt concentrations, flooding, and drought. Spartina alterniflora can also survive completely submerged in water for extended periods. It is considered a pioneer species and is the only grass with the adaptions necessary to survive harsh salt concentrations in salt marsh environments.

The present disclosure also provides an agricultural composition generated from fermenting bacteria from the Lactobacillaceae family in a growth media containing Spartina alterniflora, and such agricultural composition is configured to function as a plant biostimulant and improve agricultural efficiency. The agricultural composition of the present disclosure may improve agricultural efficiency by acting as a plant biostimulant and improving overall plant nutrition for greater plant mass, enhancing the plant's microbial population, and optimizing nutrient use efficiency. The agricultural composition of the present disclosure is capable of improving abiotic stress tolerance, such as high salt concentrations, drought, and flooding. The present disclosure relates to a plant biostimulant product that is also capable of improving the conversion of applied nutrients to plant available forms, improving conditions for better plant establishment, supporting nutrient uptake, and preventing nutrient deficiency based plant disorders.

The present disclosure addresses the need for a natural, sustainable plant biostimulant by providing an agricultural composition, and an associated method to produce the agricultural composition.

In the present disclosure, the addition of Spartina alterniflora in the growth medium for Lactobacillaceae increases plant biostimulant activity of the agricultural composition.

In some embodiments, a method may include generating an agricultural composition by fermenting at least one bacterium of Lactobacillaceae in a growth medium containing Spartina alterniflora and applying one or more filtration methods to the fermented product to isolate and extract active components for use as the agricultural composition.

In one embodiment, a method for producing an agricultural composition comprises fermenting at least one bacterium of Lactobacillaceae in a growth media containing Spartina alterniflora to produce a fermented product. The fermented product includes an agricultural composition used as a biostimulant. A filtration method is applied to the fermented product to isolate and extract the agricultural composition from the fermented product, wherein the agricultural composition contains active compounds including peptides.

In the present disclosure, the addition of Spartina alterniflora in the fermentation medium for Lactobacillaceae results in the production of bioactive components including a mixture of lipopeptides. The influence of the Spartina alterniflora when added to the growth media increases plant biostimulant activity due to a greater accumulation of bioactive compounds in the product fermented thereof compared to those from the fermentation of Lactobacillaceae in a growth media alone. The influence of Spartina alterniflora evokes an alteration in the metabolic profile of Lactobacillaceae and increases its plant biostimulant properties, allowing it to be a compelling plant biostimulant in agricultural applications.

In some embodiments, the agriculture composition may include a fermentation product derived from fermentation of gram-positive bacteria from the family of Lactobacillaceae. The gram-positive bacteria may be from the genus of Leuconostoc bacteria or Lactobacillus bacteria.

In an embodiment, a method for producing an agricultural composition includes fermenting at least once cell of Lactobacillaceae in a growth medium containing at least one stem, stalk, or seed of Spartina alterniflora to form a mixture; and filtering the mixture to isolate and extract an agricultural composition from the fermentation product.

In some embodiments, the active components in the agricultural composition may include a mixture of lipopeptides composed of 2 to 7 amino acids optionally coupled by one or more of alkyl groups, amino groups, ammonium groups, carbonyl groups, or the like. In some embodiments, an alkyl group can have a C10-C18 chain length. The molecular weight of these lipopeptides within the composition may be within 400-450 Da.

By the way of example and not limitation, the lipopeptides included in the fermentation products of Lactobacillaceae bacterium of the present disclosure can include one or more of the following Formulas 1 through 9. In some embodiments, the lipopeptides include a C₁₈H₃₈N₈O₄ lipopeptide chain, such as the following formula 1:

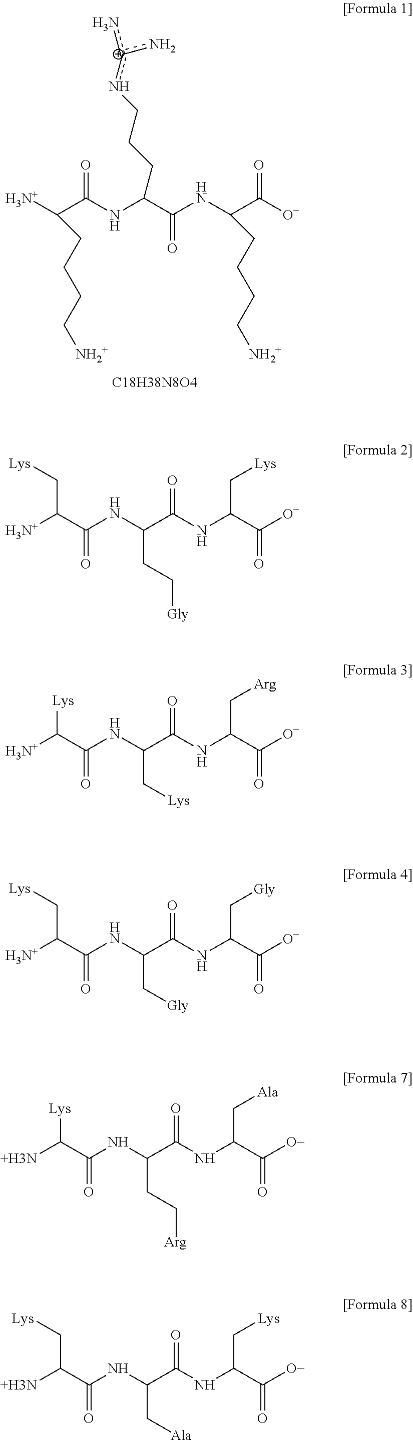

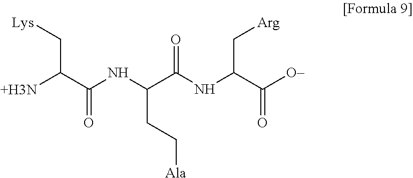

These lipopeptides may be produced naturally as a product of fermentation such as in the way described above, or synthetically derived using a synthesis method and the addition of any alkyl modification step known in the art to produce synthetic analogues of the naturally occurring lipopeptides. These synthetic analogues may be produced by any synthesis method know by those skilled in the art, including solid phase synthesis, solution phase synthesis, hybrid synthesis, Fmoc and Boc synthesis methods or a combination thereof. The addition of an alkyl modification step may include site selective modification, residue specific modification, or the addition of any alkylation method know in the art to add or modify any alkyl group on the lipopeptides chain.

The present disclosure provides an agricultural composition and an associated method for generating the agricultural composition, which is configured to function as a plant biostimulant to improve agricultural efficiency.

In one aspect, the agricultural composition may be a biostimulant or agricultural product. The biostimulant or agricultural product may be produced by fermenting at least one cell of Lactobacillaceae in a growth medium containing at least on stalk, stem, or seed of Spartina alterniflora. The biostimulant or agricultural product is then extracted from the fermentation product. The agricultural composition may be, but is not limited to, in a liquid form. In some embodiments, the agricultural composition may be a water-based composition. In one embodiment, the agricultural composition may comprise the biostimulant or agricultural product produced as discussed above, and water, wherein the biostimulant or agricultural product may have a concentration of about 0.1 wt % to about 10 wt % in the agricultural composition. It should be appreciated that the form of the agricultural composition and any compositions exemplified are not limited to the examples listed herein and any form of products are exemplified.

The application of the plant biostimulant composition may increase agricultural efficiency by producing favorable responses of targeted plants in a number of different ways. These responses may include, but are not limited to, preventing nutrient deficiency-based plant disorders, enhancing plant microbial populations, improving abiotic stress tolerance, improving conditions for better plant establishment, improving efficiency of applied fertilizers, enhancing conversion of applied nutrients to plant available forms, improving overall plant nutrients, increasing plant nutrient assimilation efficiency, increasing tolerance to sodium, and supporting nutrient uptake. The means of increasing agricultural efficiency by using the composition as a plant biostimulant may include any favorable response as a result of applying the biostimulant to the targeted plant.

The targeted plant may be, but not limited to, tomato, cannabis, lettuce, basil, cilantro, or pepper.

In some embodiments, the agricultural composition may be applied to the roots, soil, growth medium, seed, or foliar application. In one embodiment, the agricultural composition may be applied by spraying the plant above the root with the composition, applying the composition to the roots of the plant, applying the composition to a soil the plant is disposed in, applying the composition to a growth medium the plant is disposed in, applying the composition to a seed prior to growing the plant, or applying the composition to the leaves of the plant. The amount of the agricultural composition applied may depend on the type and size of the plant, and is well known by one of ordinary skill in the art.

In one aspect, a method for producing an agricultural composition is provided. At least one cell of Lactobacillaceae may be added to a growth medium containing at least one stalk, stem, or seed of Spartina alterniflora to form a mixture for fermentation.

In some embodiments, at least one stalk, stem, or seed of Spartina alterniflora is added to a growth medium, and the Spartina alterniflora and growth medium are mixed together until the Spartina alterniflora is fully dispersed in the growth medium to form a fermentation medium. The growth medium may include peptone, dextrose, ammonium citrate, sodium acetate, magnesium sulfate, manganese sulfate, potassium phosphate, or a combination thereof. In some embodiment, the Spartina alterniflora is included in an amount of about 1 wt % to about 20 wt %, and the growth medium is included in an amount of about 80 wt % to about 99 wt %, based on a total weight of the fermentation medium.

In some embodiments, at least one cell of Lactobacillaceae is added to the fermentation medium to form a mixture for fermentation. The Lactobacillaceae may be added in an amount of about 0.5 wt % to about 1.0 wt %, based on a total weight of the mixture for fermentation. In some embodiments, the Lactobacillaceae is added in an amount of about 1.0 wt % based on a total weight of the mixture for fermentation. The concentration of the bacteria in the mixture for fermentation may range from about 106 CFU/ml to about 108 CFU/ml.

The fermentation process may occur at a temperature range of about 15° C. to about 25° C. for a time period in a range of about 10 hours to about 20 hours. In other embodiments, the fermentation process may occur at a temperature in a range of about 18° C. to about 22° C. for a time period in a range of about 12 hours to 18 hours, but is not limited to these conditions. The fermentation process may be maintained at a pH in a range of about 4.0 to about 6.7. In some embodiments, the fermentation process may be maintained at a pH in a range of about 5.8 to about 6.7.

After the fermentation process, one or more filtration methods may be applied to the fermentation product to isolate and extract active compounds from the fermented product for use as an agricultural composition. In some embodiments, the extraction method may include, but not limited to, tangential flow filtration followed by sterile filtration method. The active compounds may include a mixture of lipopeptides configured to function as a plant biostimulant to improve agricultural productivity. In some embodiments, the mixture of lipopeptides may be composed of 2-7 amino acids with alkyl groups of a C1-C18 chain length. The molecular weight of these peptides within the composition may be within 400-450 Da and can include a C₁₈H₃₈N₈O₄ lipopeptide chain.

### EXAMPLES

Hereinafter, the present disclosure is specifically described by way of examples, but the scope of the present application is not limited by the following examples. The agricultural composition was prepared according to the following examples.

### Example—Leuconostoc kimchii and Growth Media Containing Spartina alterniflora Ferment Extract

An agricultural composition of Example was prepared by fermenting Leuconostoc kimchi in a growth media containing Spartina alterniflora. The growth media was De Man, Rogosa, and Sharpe (MRS) broth. The contents of the MRS broth included peptone, dextrose, ammonium citrate, sodium acetate, magnesium sulfate, manganese sulfate, potassium phosphate and a combination thereof. The Spartina alterniflora and growth medium were mixed together until the Spartina alterniflora was fully dispersed in the growth medium to form a fermentation medium, and the Leuconostoc kimchi was added to form a mixture for fermentation, and this mixture comprised 1 wt % of the Leuconostoc kimchi, 10 wt % of the Spartina alterniflora, and 89 wt % of the MRS broth. The mixture was fermented at 20° C. for 12 hours to 18 hours at a pH range of 5.8 to 6.7. After 12 to 18 hours, the fermentation product was filtered using tangential flow filtration followed by sterile filtration to collect the ferment extract to be used as the agricultural composition.

### Comparative Example—Leuconostoc kimchi and Growth Media Ferment Extract

An agricultural composition of Comparative Example was prepared in a similar manner to the Example, except omitting the Spartina alterniflora. The agricultural composition was prepared by fermenting Leuconostoc kimchi in a growth media (MRS broth). Leuconostoc kimchi was fermented at 20° C. for 12 to 18 hours at a pH range of 5.8 to 6.7. After 12 hours to 18 hours, the fermentation product was filtered using tangential flow filtration followed by sterile filtration to collect the ferment extract to be used as the agricultural composition.

### Experimental Example—Biostimulant

A biostimulant assay was conducted to evaluate the ability of the agricultural compositions to act as a plant biostimulant. This experiment examined the results of foliar application of the agricultural compositions on tomato plants. The results were examined for the following preparations: Experimental Example 1: an agricultural composition having Leuconostoc kimchii and growth media containing Spartina alterniflora ferment extract (Example), Experimental Example 2: an agricultural composition having Leuconostoc kimchii ferment and growth media extract (Comparative Example).

The materials used in the testing included: two sets of four 3-gallon buckets, clear vinyl tubing, aquarium pump, air stone, Polyvinyl chloride (PVC) pipe, pea gravel, lava rock, deionized water, Tomato seedlings (Bonnie Plants)

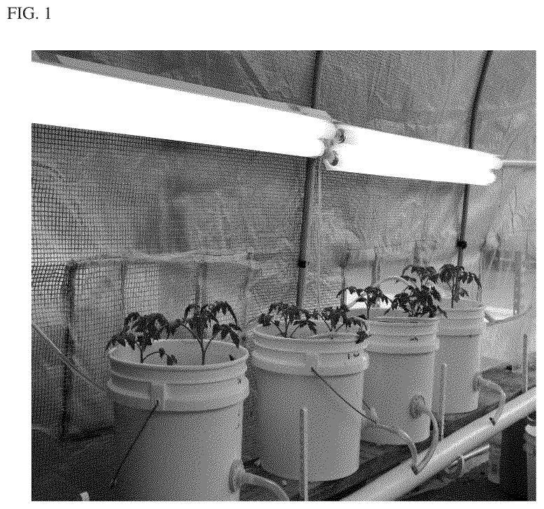

The experiment began with the construction of a bato bucket system (depicted in FIG. 1), where four three-gallon buckets were used as containers. Each bucket had a drainage hole of about 2 inches from the bottom of the bucket. The buckets were filled with a mix of lava rock and pea pebbles as a growth medium. Two Tomato seedlings (Bonnie Plants) were transferred to each bucket and allowed to established growth for one week, where water lines run along the tops of each bucket supplied nutrients on a watering schedule was 30 minutes, three times per day and placed in full sunlight. The plants were grown in a greenhouse during early spring to early summer, mornings in low 70s° F., mid-day upper 80s° F. in full sunlight. Two bato bucket systems of the same kind were constructed.

Media/Buffers of this experiment included: Deionized water for each of Experimental Examples 1-2, wherein Experimental Examples 1-2 contain 0.1 wt % of Example and Comparative Example, respectively.

Initial weights were measured for each plant. In the first bato bucket system, all plants were watered 30 minutes per day, three times daily. For the first three buckets, the plants were also sprayed with the compositions of Experimental Example 1 in weekly, bi-weekly, and daily increments whereas the plant in the fourth bucket was not treated with any agricultural composition (Control). In the second bato bucket system, all plants were watered 30 minutes per day, three times daily. For the first three buckets, the plants were also sprayed with the compositions of Experimental Example 2 in weekly, bi-weekly, and daily increments in the three buckets whereas the plant in the fourth bucket was not treated with any agricultural composition (Control). The plants were sprayed with the compositions of Experimental Examples 1 or 2 mid-morning on the same days, and they were sprayed thoroughly until the solution ran off the leaves, and normally about 2-20 sprays were applied. At the conclusion of the study, the plants were removed from the buckets and measured for final weight.

Over the course of seven weeks, the plants with the treatments of the agricultural composition of Experimental Example 1 having Leuconostoc kimchi and growth media containing Spartina alterniflora ferment extract (Example) performed better than those with the treatments of the agricultural composition of Experimental Example 2 having the Leuconostoc kimchii and growth media ferment extract (Comparative Example) or the untreated Controls. The plants treated with the agricultural composition of Experimental Example 1 having Leuconostoc kimchii and growth media containing Spartina alterniflora ferment extract (Example) accumulated more biomass and had larger root systems than those with the treatments of the agricultural composition of Experimental Example 2 having the Leuconostoc kimchii and growth media ferment extract (Comparative Example) or the untreated Controls.

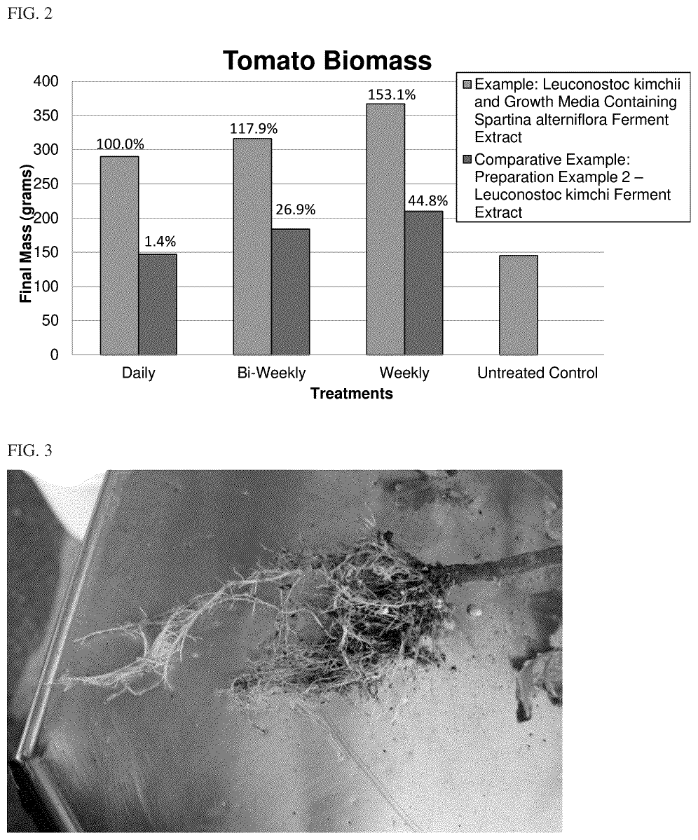

Among them, the plant with the weekly treatment of the agricultural composition of Experimental Example 1 having Leuconostoc kimchii and growth media containing Spartina alterniflora ferment extract (Example) accumulated the most biomass, with an increase of 153.1% compared to the untreated Control as shown in FIG. 2.

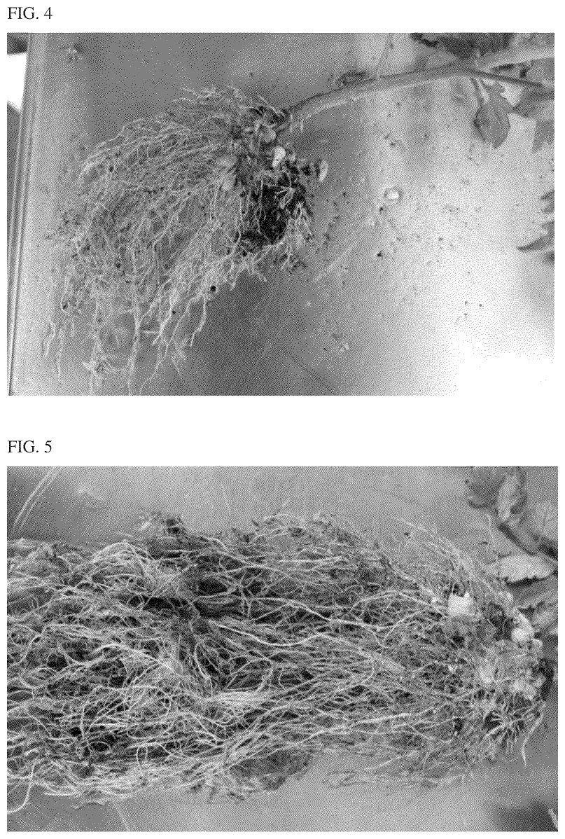

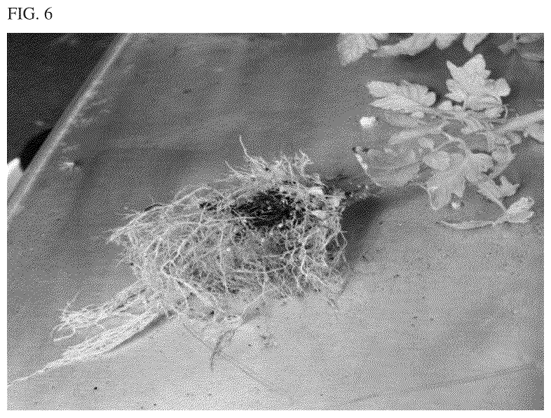

The results of the biostimulant assay demonstrated the effectiveness of the agricultural composition having Leuconostoc kimchii and growth media containing Spartina alterniflora ferment extract as a plant biostimulant. When tomato plants were sprayed with foliar application of the disclosed agricultural composition, it improved overall plant mass by improved nutrient uptake. FIG. 3 depicts root size after daily treatment with Experimental Example 1. FIG. 4 depicts root size after bi-weekly treatments with Experimental Example 1. FIG. 5 displays root size after weekly treatment with Experimental Example 1. FIG. 6 depicts the root size for the untreated Control. This increase can improve the plant's ability to absorb and process nutrients, therefore increasing the overall health of the plant.

Weekly treatment application appeared most effective, however all application frequencies of the agricultural composition having Leuconostoc kimchii and growth media containing Spartina alterniflora ferment extract (Example) increased the biomass the plant when compared to untreated Controls.

The improvement in root formation and biomass demonstrated that the disclosed agricultural composition can improve plant vigor and agricultural efficiency. The result of this study indicates that the Leuconostoc kimchii fermentation extract is most effective as a plant biostimulant when it is fermented in a growth media containing Spartina alterniflora compared to the Leuconostoc kimchii ferment extract in a growth media alone.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of producing a plant biostimulant, comprising:
adding at least one stalk, stem, or seed of Spartina alterniflora to a growth media to form a fermentation medium,
adding at least one cell of Lactobacillaceae to the fermentation medium to form a mixture for fermentation,
fermenting the Lactobacillaceae in the mixture for fermentation to produce a fermentation product; and
applying a filtration method to the fermentation product to extract a plant biostimulant from the fermentation product.

2. The method of claim 1, the plant biostimulant comprises lipopeptides having 2-7 amino acids optionally coupled with an alkyl group having a C1-C18 chain.

3. The method of claim 1, wherein the growth media comprises at least one selected from peptone, dextrose, ammonium citrate, sodium acetate, magnesium sulfate, manganese sulfate, or potassium phosphate.

4. The method of claim 1, wherein the fermentation step is performed at a temperature in a range of about 15° C. to about 25° C.

5. The method of claim 3, wherein the fermentation step is performed at the temperature of about 20° C.

6. The method of claim 1, wherein the fermentation step is performed for a time period of 12 hours to about 18 hours.

7. The method of claim 1, wherein the fermentation step is performed at a pH range of about 4.0 to 6.7.

8. The method of claim 1, wherein the Spartina alterniflora is included in an amount of about 1 wt % to about 20 wt %, based on a total amount of the fermentation medium.

9. The method of claim 1, wherein the Lactobacillaceae is present in an amount of about 0.5 wt % to about 1 wt %, based on a total amount of the mixture for fermentation.

10. The method of claim 1, where the Lactobacillaceae is present in a concentration of about 106 CFU/ml to about 108 CFU/ml in the mixture for fermentation.

11. The method of claim 9, wherein the lipopeptides are one or more selected from the following compounds:

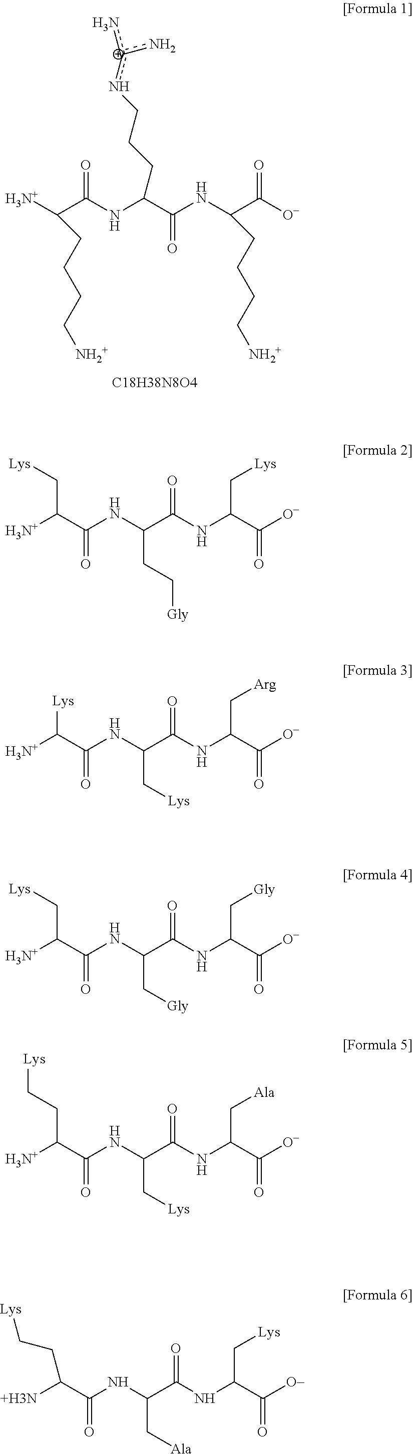

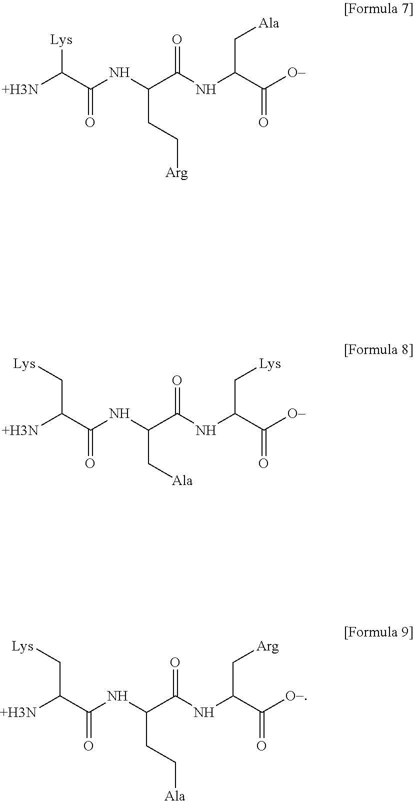

12. A method of increasing an overall plant mass by improving nutrient uptake, comprising:
applying a plant biostimulant to a plant, wherein the plant biostimulant comprises an extract obtained from a fermentation product of a gram-positive bacteria from the family of Lactobacillaceae and a growth media comprising Spartina alterniflora.

13. The method of claim 12, wherein the extract comprises lipopeptides having 2 to 7 amino acids.

14. The method of claim 12, wherein the applying of the plant biostimulant comprises:
at least one of spraying the plant above the root with the plant biostimulant, applying the plant biostimulant to the roots of the plant, applying the plant biostimulant to a soil the plant is disposed in, applying the plant biostimulant to a medium the plant is disposed in, applying the plant biostimulant to a seed prior to growing the plant, or applying the plant biostimulant to leaves of the plant.

15. A plant biostimulant, comprising:
an extract obtained from a fermentation product of a gram-positive bacteria from the family of Lactobacillaceae and a growth media comprising Spartina alterniflora.

16. The plant biostimulant of claim 15, wherein the extract comprises lipopeptides having 2 to 7 amino acid.

17. The plant biostimulant of claim 16, wherein the lipopeptides are one or more selected from the following compounds: 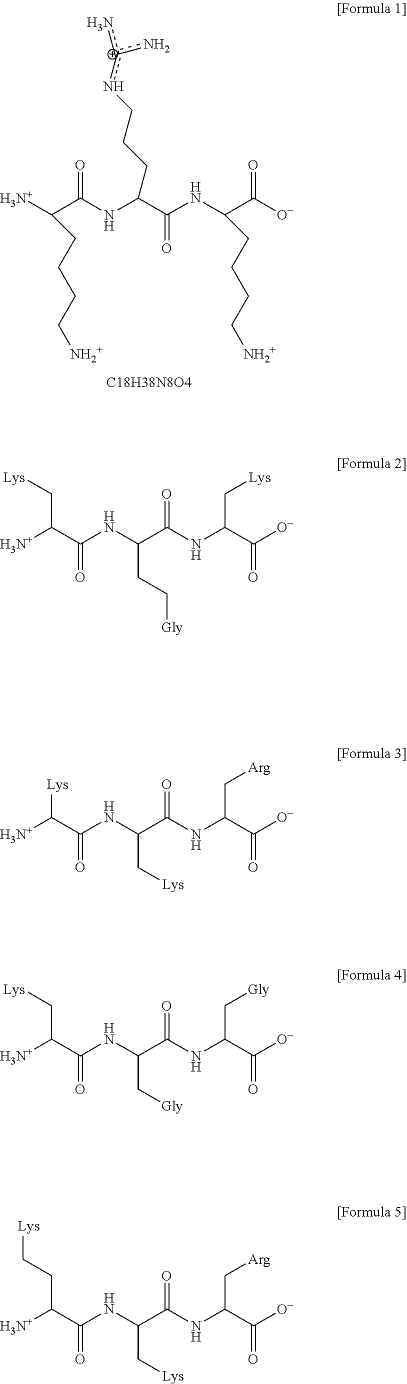 [chemical structure]
